(19) United States
(12) Patent Application Publication
(10) Pub. No.: US 2015/0011728 A1
(43) Pub. Date: Jan. 8, 2015

(54) REVERSIBLY CROSSLINKED HELICAL HYDROGEN BOND SURROGATE MACROCYCLES

(71) Applicants: Paramjit S. Arora, Huntington, NY (US); Stephen E. Miller, Rome, NY (US)

(72) Inventors: Paramjit S. Arora, Huntington, NY (US); Stephen E. Miller, Rome, NY (US)

(73) Assignee: NEW YORK UNIVERSITY, New York, NY (US)

(21) Appl. No.: 14/380,708

(22) PCT Filed: Feb. 22, 2013
(86) PCT No.: PCT/US2013/027324

§ 371 Date: Aug. 22, 2014

Related U.S. Application Data
(60) Provisional application No. 61/602,017, filed on Feb. 22, 2012.

(51) Int. Cl. C07K 7/64

(52) U.S. Cl. CPC C07K 7/64
USPC 530/321

(57) ABSTRACT

The present invention relates to peptides having one or more stable, reversibly and internally-constrained HBS α-helices.

This application claims the benefit of U.S. Provisional Patent Application Ser. No. 61/602,017, filed Feb. 22, 2012, which is hereby incorporated by reference in its entirety.

### STATEMENT AS TO FEDERALLY SPONSORED RESEARCH

This invention was made with U.S. Government support under Grant No. R01GM073943, awarded by the National Institutes of Health, and Grant. Nos. CHE1027009 and CHE-0958457, awarded by the National Science Foundation. The U.S. Government has certain rights in this invention.

### BACKGROUND OF THE INVENTION

Methods that provide reversible control over protein secondary structure formation have proven to be useful for probing protein structure and interactions, and can be suitable in therapeutic, diagnostic, or other applications. The invention addresses these and other needs.

### SUMMARY OF THE INVENTION

Provided herein are methods of synthesizing a stabilized helical peptidomimetic macrocycle comprising: providing a peptidomimetic precursor comprising two thiol groups; contacting the precursor with a reagent capable of inducing a reaction between said two thiol groups, said reaction resulting in formation of a disulfide covalent bond; wherein said contacting step results in cyclization of the precursor to form said stabilized helical peptidomimetic macrocycle.

In some embodiments, said stabilized helical peptidomimetic macrocycle comprises a structure of formula:

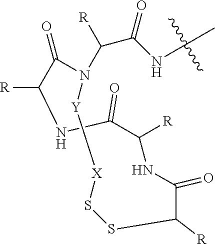

wherein each R is independently an amino acid side chain, and X—Y is a crosslinker moiety.

For example, the peptidomimetic macrocycle comprises an α-helix. In some embodiments, the stabilized peptidomimetic macrocycle has higher α-helicity compared to a corresponding non-macrocyclic polypeptide. In other embodiments, the stabilized peptidomimetic macrocycle has higher α-helicity compared to the peptidomimetic precursor. For example, the α-helicity is measured by circular dichroism.

In some embodiments, the peptidomimetic macrocycle exhibits increased resistance to proteolytic degradation compared to a corresponding non-macrocyclic polypeptide. In other embodiments, the peptidomimetic macrocycle exhibits increased biological activity compared to a corresponding non-macrocyclic polypeptide.

In some embodiments, the peptidomimetic precursor is prepared by solid phase peptide synthesis resin. For example, the peptidomimetic precursor is attached to a solid phase peptide synthesis resin during the contacting step. In other embodiments, the peptidomimetic precursor is not attached to a solid phase peptide synthesis resin during the contacting step.

In some embodiments, the contacting step takes place in a solvent. For example, the solvent is an aqueous solvent. In some embodiments, the solvent comprises DMSO and/or TFE. In some embodiments, the peptidomimetic macrocycle is purified after the contacting step.

In some embodiments, a peptidomimetic macrocycle comprises a structure of formula:

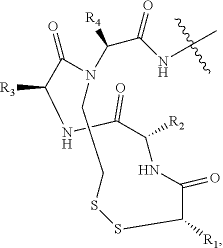

wherein R₁, R₂, R₃ and R₄ are each independently an amino acid side chain. In other embodiments, a stabilized helical peptidomimetic macrocycle comprises a structure of formula:

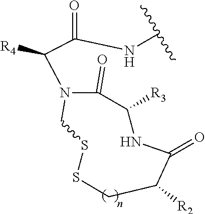

wherein R₁, R₂, R₃ and R₄ are each independently an amino acid side chain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows (a) hydrogen bond surrogate (HBS) α-helices which feature a covalent bond in place of the intramolecular hydrogen bond between the i and i+4 residues. In dsHBS α-helices the main chain hydrogen bond is replaced with a disulfide linkage, and can be reversibly formed from a bis-thiol peptide (b).

FIG. 2 shows circular dichroism spectra of dsHBS 2 in mixtures of PBS and TFE.

FIG. 3 shows a scheme for the solid phase synthesis of macrocycles.

FIG. 4 shows DMSO-mediated conversion of bis-thiol precursor 1 to a macrocycle.

FIG. 5 shows circular dichroism spectra of peptides 1, 2 and 6. The CD spectra were obtained in phosphate buffered saline. TCEP=tris(2-carboxyethyl)phosphine. Calculated % helicity values: 1: 22%, 2: 55%, 2+TCEP: 22%, and 6: 14%.

FIG. 6 shows short-range (dashed arrows) and medium-range (black arrows) NOE's observed for dsHBS 2. 2D NMR spectra were acquired in 20% trifluoroethanol-d3/PBS at 20° C.

FIG. 7 shows a ¹H NMR spectrum of dsHBS 2 in 20% trifluoroethanol-d3/PBS.

FIG. 8 shows ¹H NMR assignments and chemical shifts (δ, ppm) for dsHBS 2 in 20% trifluoroethanol-d3/PBS. *αCH for glycine could not be unambiguously assigned.

FIG. 9 shows a complete NOESY correlation chart for dsHBS 2. The glycine-3 residue is N-alkylated. Filled rectangles indicate relative intensities of NOE cross-peaks. Empty rectangles indicate NOEs that could not be unambiguously assigned because of overlapping signals.

FIG. 10 shows a NOESY spectrum for dsHBS 2 in 20% TFE/PBS.

FIG. 11 shows the NH region of the NOESY spectrum for dsHBS 2.

FIG. 12 shows a TOCSY spectrum for dsHBS 2 in 20% TFE/PBS.

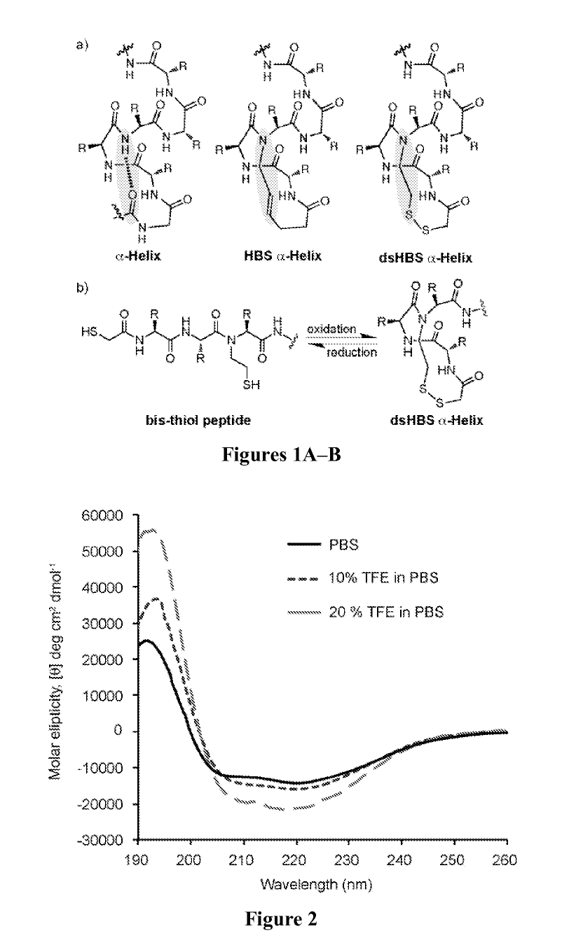

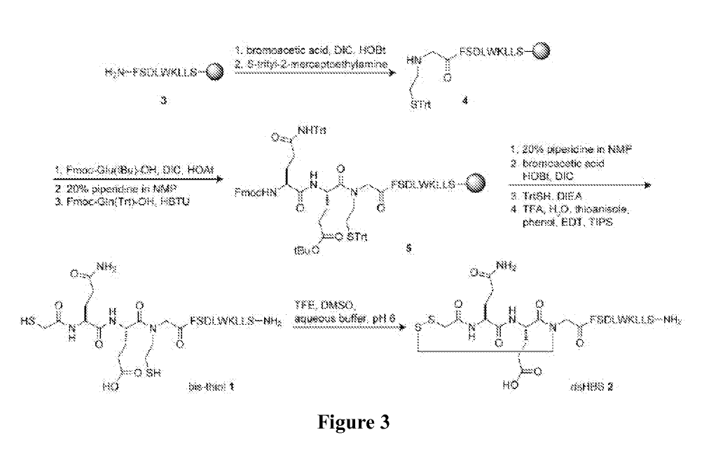

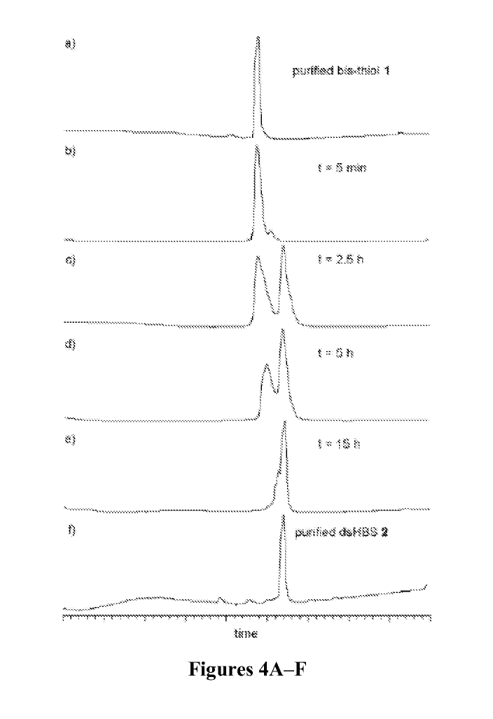

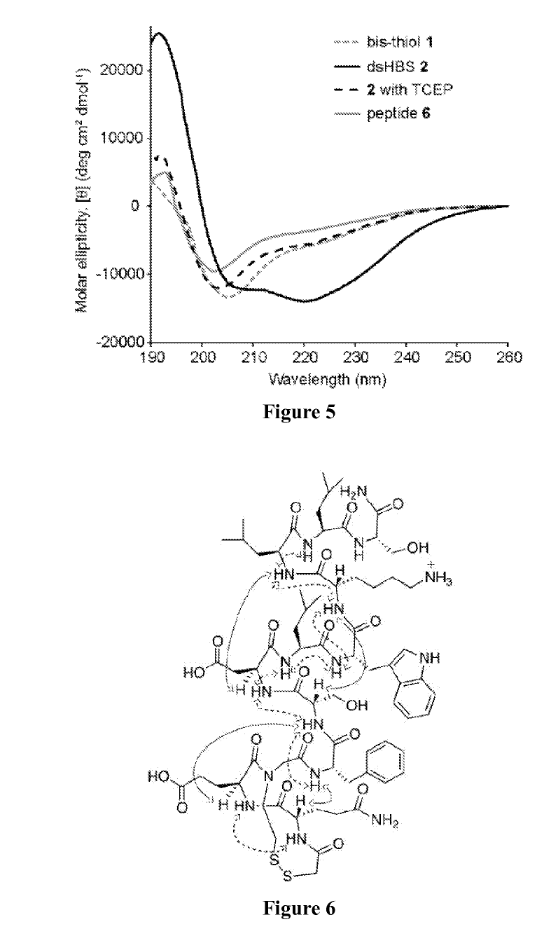

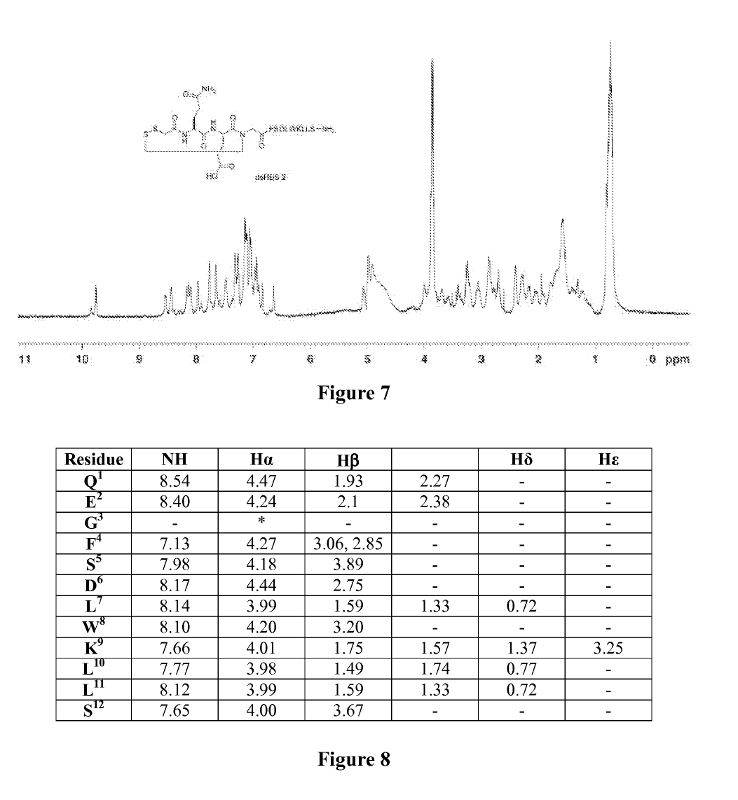

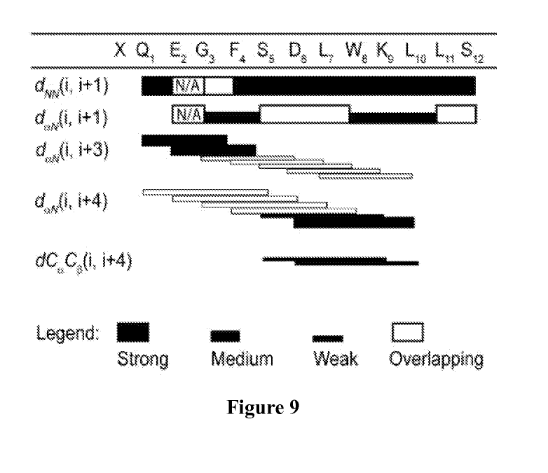

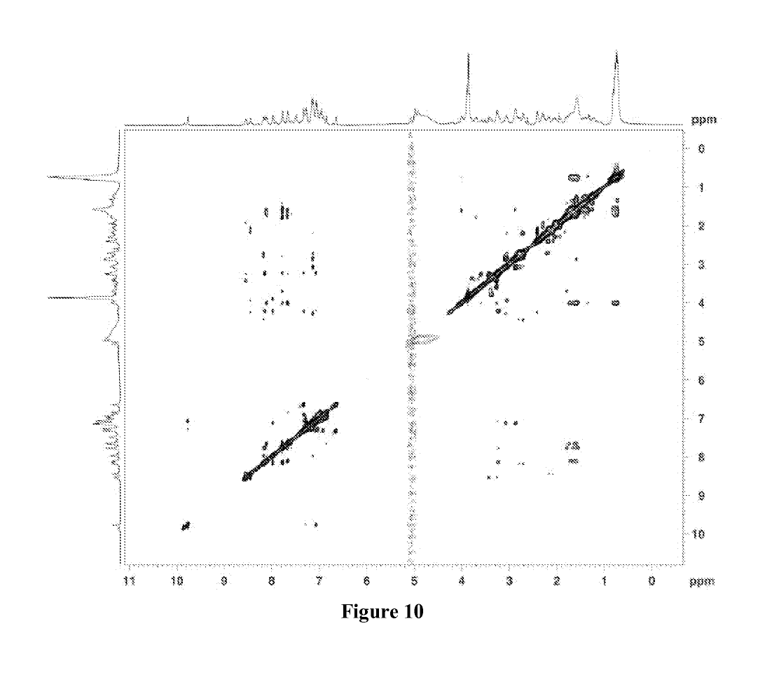

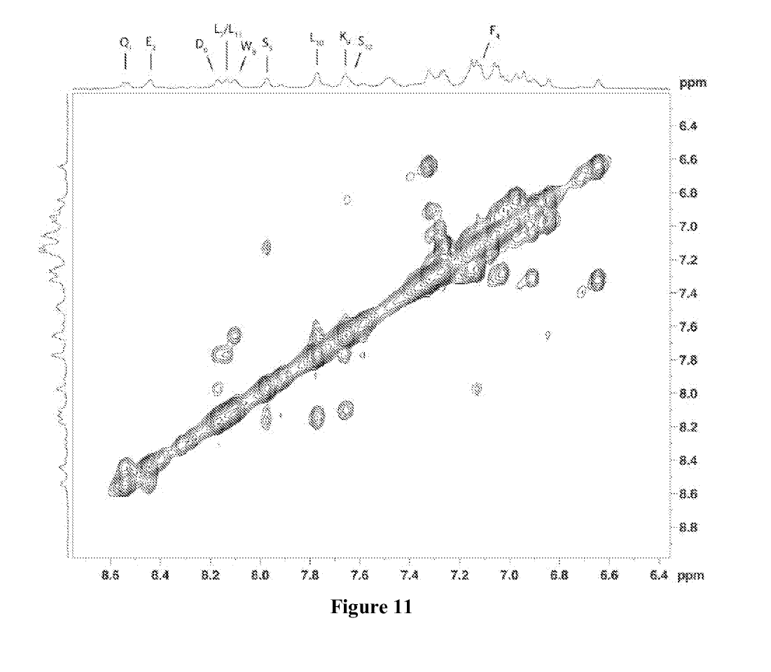

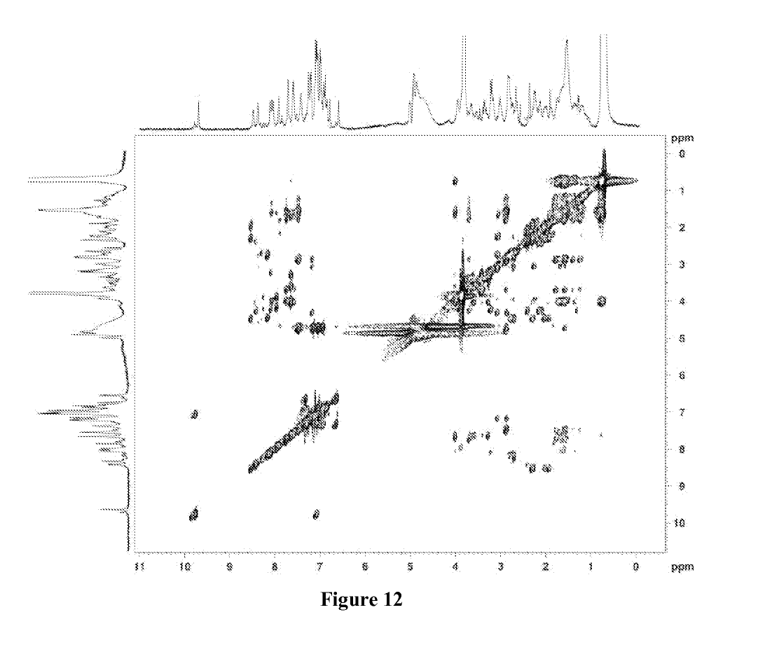

### DETAILED DESCRIPTION OF THE INVENTION

The present invention relates to hydrogen bond surrogate (“HBS”)-derived helices, including α-helices. These HBS helices can potentially function as in vivo inhibitors of biological protein interactions.

Provided herein are peptides having one or more stable, internally-constrained HBS α-helices, where the peptide mimics at least a portion of a protein capable of interacting with another protein. For example, the peptide mimics an alpha-helical portion of the protein capable of interacting with another protein. The term “mimic” refers to the ability of a composition of the invention to effect a similar activity as a natural protein such as its partner. A “mimic” encompasses both functional and structural mimics of such proteins. For example, the mimic is a protein which shares a certain percent homology (e.g. 60%, 70%, 80%, 85%, 90%, or 95% homology) with the target protein. Alternatively, the mimic is derived from a different sequence that nevertheless is capable of interacting with its binding partner in a functionally similar manner, for example by interacting with the same active site.

As will be apparent to one of ordinary skill in the art, the methods of the present invention may be used to prepare peptides having highly stabilized, internally-constrained α-helices. The constraint may be placed anywhere within the peptide, not just at the N-terminus. For example, a compound provided herein may comprise a structure of the formula

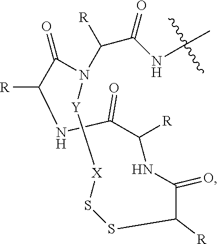

wherein each R is independently an amino acid side chain, and X—Y is a crosslinker moiety.

For example, the compound provided herein comprises a structure of the formula:

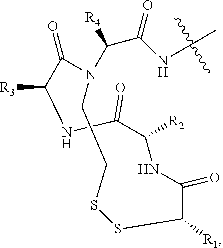

wherein R₁, R₂, R₃ and R₄ are each independently an amino acid side chain.

In other embodiments, the macrocycles disclosed herein comprise a structure of the formula:

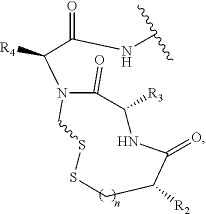

wherein R₂, R₃ and R₄ are each independently an amino acid side chain.

The peptides produced according to the methods of the present invention may, for example, be less than 40, 30, 25, 20, or 15 amino acids, including, for example, less than 10 amino acid residues.

HBS α-helices of the present invention are obtained by replacing an N-terminal main-chain i and i+4 hydrogen bond with a disulfide bond. The hydrogen bond surrogate pre-organizes an α-turn and stabilizes the peptide sequence in an α-helical conformation.

In another aspect, preparing a compound of the invention involves providing a bis-thiol peptide precursor compound and promoting disulfide bond formation to result in a stable, internally-constrained helix, such as an α-helix. Other secondary structures, for example helices such as 3₁₀ helices, can also be prepared using the methods disclosed herein.

The reactions disclosed herein may, for example, be carried out on a solid support. Suitable solid supports include particles, strands, precipitates, gels, sheets, tubing, spheres, containers, capillaries, pads, slices, films, plates, slides, discs, membranes, etc. These solid supports can be made from a wide variety of materials, including polymers, plastics, ceramics, polysaccharides, silica or silica-based materials, carbon, metals, inorganic glasses, membranes, or composites thereof. The substrate is preferably flat but may take on a variety of alternative surface configurations. For example, the substrate may contain raised or depressed regions on which the synthesis takes place. The substrate and its surface preferably form a rigid support on which to carry out the reactions described herein. Other substrate materials will be readily apparent to those of ordinary skill in the art upon review of this disclosure.

As will be apparent to one of ordinary skill in the art, administering may be carried out using generally known methods.

Administration can be accomplished either via systemic administration to the subject or via targeted administration to affected cells. Exemplary routes of administration include, without limitation, by intratracheal inoculation, aspiration, airway instillation, aerosolization, nebulization, intranasal instillation, oral or nasogastric instillation, intraperitoneal injection, intravascular injection, topically, transdermally, parenterally, subcutaneously, intravenous injection, intra-arterial injection (such as via the pulmonary artery), intramuscular injection, intrapleural instillation, intraventricularly, intralesionally, by application to mucous membranes (such as that of the nose, throat, bronchial tubes, genitals, and/or anus), or implantation of a sustained release vehicle.

Typically, the peptide of the present invention will be administered to a mammal as a pharmaceutical formulation that includes the therapeutic agent and any pharmaceutically acceptable adjuvants, carriers, excipients, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of therapeutic agent together with the adjuvants, carriers and/or excipients. The amount of active compound in such therapeutically useful compositions is such that a suitable dosage unit will be obtained.

The agents may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of the agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient(s), sucrose as a sweetening agent, methyl and propylparabens as preservatives, a dye, and flavoring such as cherry or orange flavor.

The agents may also be administered parenterally. Solutions or suspensions of the agent can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents according to this aspect of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The agents of the present invention may be administered directly to a targeted tissue, e.g., tissue that is susceptible to the condition to be treated. Additionally and/or alternatively, the agent may be administered to a non-targeted area along with one or more agents that facilitate migration of the agent to (and/or uptake by) a targeted tissue, organ, or cell. As will be apparent to one of ordinary skill in the art, the therapeutic agent itself be modified to facilitate its transport to (and uptake by) the desired tissue, organ, or cell.

Exemplary delivery devices include, without limitation, nebulizers, atomizers, liposomes, transdermal patches, implants, implantable or injectable protein depot compositions, and syringes. Other delivery systems which are known to those of skill in the art can also be employed to achieve the desired delivery of the therapeutic agent to the desired organ, tissue, or cells in vivo to effect this aspect of the present invention.

Any suitable approach for delivery of the agents can be utilized to practice this aspect of the present invention. Typically, the agent will be administered to a patient in a vehicle that delivers the agent(s) to the target cell, tissue, or organ.

One approach for delivering agents into cells involves the use of liposomes. Basically, this involves providing a liposome which includes agent(s) to be delivered, and then contacting the target cell, tissue, or organ with the liposomes under conditions effective for delivery of the agent into the cell, tissue, or organ.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., each of which is hereby incorporated by reference in its entirety.

These liposomes can be produced such that they contain, in addition to the therapeutic agents of the present invention, other therapeutic agents, such as anti-inflammatory agents, which would then be released at the target site (e.g., Wolff et al., “The Use of Monoclonal Anti-Thy1 IgG1 for the Targeting of Liposomes to AKR-A Cells in Vitro and in Vivo,” Biochim. Biophys. Acta 802:259-73 (1984), which is hereby incorporated by reference in its entirety).

An alternative approach for delivery of proteins or polypeptide agents (e.g., peptides of the present invention) involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptide agents involves preparation of chimeric proteins according to U.S. Pat. No. 5,817,789 to Heartlein et al., which is hereby incorporated by reference in its entirety. The chimeric protein can include a ligand domain and the polypeptide agent (e.g., the artificial α-helix of the present invention). The ligand domain is specific for receptors located on a target cell. Thus, when the chimeric protein is delivered intravenously or otherwise introduced into blood or lymph, the chimeric protein will adsorb to the targeted cell, and the targeted cell will internalize the chimeric protein.

Administration can be carried out as frequently as required and for a duration that is suitable to provide effective treatment. For example, administration can be carried out with a single sustained-release dosage formulation or with multiple daily doses.

The amount to be administered will, of course, vary depending upon the treatment regimen. Generally, an agent is administered to achieve an amount effective for an improvement in the state of the patient (i.e., a therapeutically effective amount). Thus, in the case of cancer, a therapeutically effective amount can be an amount which is capable of at least partially decreasing the size of a tumor, decreasing the number of cancerous cells in the body, or slowing the increase in number of cancer cells in the body. The dose required to obtain an effective amount may vary depending on the agent, formulation, cancer, and individual to whom the agent is administered.

Determination of effective amounts may also involve in vitro assays in which varying doses of agent are administered to cells in culture and the concentration of agent effective for inhibiting growth of cancer cells is determined in order to calculate the concentration required in vivo. Effective amounts may also be based on in vivo animal studies. A therapeutically effective amount can be determined empirically by those of skill in the art.

### Methods of Treatment

In some embodiments, the compounds of the invention is used to treat, prevent, and/or diagnose cancers and neoplastic conditions. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the compounds are novel therapeutic agents for controlling breast cancer, ovarian cancer, colon cancer, lung cancer, metastasis of such cancers and the like.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991), Crit Rev. Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

In some embodiments, the peptides of the invention are used to treat a cancer mediated by a mutated Ras protein. Cancers known to frequently involve such mutations include, but are not limited to, non-small-cell lung cancer (adenocarcinoma), colorectal cancer, pancreatic cancer, thyroid cancers (e.g. follicular, undifferentiated papillary or papillary), seminoma, melanoma, bladder cancer, liver cancer, kidney cancer, myelodysplastic syndrome, and acute myelogenous leukemia.

### Breast Cancer

In one aspect, the invention provides methods of treating breast cancer by administering the compounds of the invention. Breast cancer includes invasive breast carcinomas, such as invasive ductal carcinoma, invasive lobular carcinoma, tubular carcinoma, invasive cribriform carcinoma, medullary carcinoma, mucinous carcinoma and other tumours with abundant mucin, cystadenocarcinoma, columnar cell mucinous carcinoma, signet ring cell carcinoma, neuroendocrine tumours (including solid neuroendocrine carcinoma, atypical carcinoid tumour, small cell/oat cell carcinoma, or large cell neuroendocrine carcioma), invasive papillary carcinoma, invasive micropapillary carcinoma, apocrine carcinoma, metaplastic carcinomas, pure epithelial metaplastic carciomas, mixed epithelial/mesenchymal metaplastic carcinomas, lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma or bilateral breast carcinoma; mesenchymal tumors such as haemangioma, angiomatosis, haemangiopericytoma, pseudoangiomatous stromal hyperplasia, myofibroblastoma, fibromatosis (aggressive), inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma, or leiomysarcoma; myoepithelial lesions such as myoepitheliosis, adenomyoepithelial adenosis, adenomyoepithelioma, or malignant myoepithelioma; fibroepithelial tumours such as fibroadenoma, phyllodes tumour, low grade periductal stromal sarcoma, or mammary hamartoma; and tumours of the nipple such as nipple adenoma, syringomatous adenoma, or Paget's disease of the nipple.

Treatment of breast cancer may be effected in conjunction with any additional therapy, such as a therapy that is part of the standard of care. A surgical technique such as lumpectomy or mastectomy may be performed prior to, during, or following treatment with the compounds of the invention. Alternatively, radiation therapy may be used for the treatment of breast cancer in conjunction with the compounds of the invention. In other cases, the compounds of the invention are administered in combination with a second therapeutic agent. Such an agent may be a chemotherapeutic agent such as an individual drug or combination of drugs and therapies. For example, the chemotherapeutic agent can be an adjuvant chemotherapeutic treatment such as CMF (cyclophosphamide, methotrexate, and 5-fluorouracil); FAC or CAF (5-fluorouracil, doxorubicin, cyclophosphamide); AC or CA (doxorubicin and cyclophosphamide); AC-Taxol (AC followed by paclitaxel); TAC (docetaxel, doxorubicin, and cyclophosphamide); FEC (5-fluorouracil, epirubicin and cyclophosphamide); FECD (FEC followed by docetaxel); TC (docetaxel and cyclophosphamide). In addition to chemotherapy, trastuzumab may also be added to the regimen depending on the tumor characteristics (i.e. HER2/neu status) and risk of relapse. Hormonal therapy may also be appropriate before, during or following chemotherapeutic treatment. For example, tamoxifen may be administered or a compound in the category of aromatase inhibitors including, but not limited to aminogluthetimide, anastrozole, exemestane, formestane, letrozole, or vorozole. In other embodiments, an antiangiogenic agent may be used in combination therapy for the treatment of breast cancer. The antiangiogenic agent may be an anti-VEGF agent including, but not limited to bevacizumab.

### Ovarian Cancer

In another aspect, the compounds of the invention may be used to treat ovarian cancer. Ovarian cancers include ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

The compounds of the invention may be administered in conjunction with a second therapy such as a therapy that is part of the standard of care. Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpigectomy.

Anti-cancer drugs that may be used include cyclophosphamide, etoposide, altretamine, and ifosfamide. Hormone therapy with the drug tamoxifen may be used to shrink ovarian tumors. Radiation therapy may be external beam radiation therapy and/or brachytherapy.

### Prostate Cancer

In another aspect, the compounds of the invention may be used to treat prostate cancer. Prostate cancers include adenocarcinomas and metastasized adenocarcinomas. The compounds of the invention may be administered in conjunction with a second therapy such as a therapy that is part of the standard of care. Treatment for prostate cancer may involve surgery, radiation therapy, High Intensity Focused Ultrasound (HIFU), chemotherapy, cryosurgery, hormonal therapy, or any combination thereof. Surgery may involve prostatectomy, radical perineal prostatectomy, laparoscopic radical prostatectomy, transurethral resection of the prostate or orchiectomy. Radiation therapy may include external beam radiation therapy and/or brachytherapy. Hormonal therapy may include orchiectomy; administration of antiandrogens such as flutamide, bicalutamide, nilutamide, or cyproterone acetate; medications which inhibit the production of adrenal androgens such as DHEA, such as ketoconazole and aminoglutethimide; and GnRH antagonists or agonists such as Abarelix (Plenaxis®), Cetrorelix (Cetrotide®), Ganirelix (Antagon®), leuprolide, goserelin, triptorelin, or buserelin. Treatment with an anti-androgen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy includes, but is not limited to, administration of docetaxel, for example with a corticosteroid such as prednisone. Anti-cancer drugs such as doxorubicin, estramustine, etoposide, mitoxantrone, vinblastine, paclitaxel, carboplatin may also be administered to slow the growth of prostate cancer, reduce symptoms and improve the quality of life. Additional compounds such as bisphosphonate drugs may also be administered.

### Renal Cancer

In another aspect, the compounds of the invention may be used to treat renal cancer. Renal cancers include, but are not limited to, renal cell carcinomas, metastases from extra-renal primary neoplasms, renal lymphomas, squamous cell carcinomas, juxtaglomerular tumors (reninomas), transitional cell carcinomas, angiomyolipomas, oncocytomas and Wilm's tumors. The compounds of the invention may be administered in conjunction with a second therapy such as a therapy that is part of the standard of care. Treatment for renal cancer may involve surgery, percutaneous therapies, radiation therapies, chemotherapy, vaccines, or other medication. Surgical techniques useful for treatment of renal cancer in combination with the compounds of the invention include nephrectomy, which may include removal of the adrenal gland, retroperitoneal lymph nodes, and any other surrounding tissues affected by the invasion of the tumor. Percutaneous therapies include, for example, image-guided therapies which may involve imaging of a tumor followed by its targeted destruction by radiofrequency ablation or cryotherapy. In some cases, other chemotherapeutic or other medications useful in treating renal cancer may be alpha-interferon, interleukin-2, bevacizumab, sorafenib, sunitib, temsirolimus or other kinase inhibitors.

### Pancreatic Cancer

In other aspects, the invention provides methods of treating pancreatic cancer by administering compounds of the invention, such as a pancreatic cancer selected from the following: an epitheliod carcinoma in the pancreatic duct tissue and an adenocarcinoma in a pancreatic duct. The most common type of pancreatic cancer is an adenocarcinoma, which occurs in the lining of the pancreatic duct. Possible treatments available for pancreatic cancer include surgery, immunotherapy, radiation therapy, and chemotherapy. Possible surgical treatment options include a distal or total pancreatectomy and a pancreaticoduodenectomy (Whipple procedure). Radiation therapy may be an option for pancreatic cancer patients, specifically external beam radiation where radiation is focused on the tumor by a machine outside the body. Another option is intraoperative electron beam radiation administered during an operation. Chemotherapy may also be used to treat pancreatic cancer patients. Suitable anti-cancer drugs include, but are not limited to, 5-fluorouracil (5-FU), mitomycin, ifosfamide, doxorubicin, streptozocin, chlorozotocin, and combinations thereof. The methods provided by the invention can provide a beneficial effect for pancreatic cancer patients, by administration of a polypeptide of the invention or a combination of administration of a compound and surgery, radiation therapy, or chemotherapy.

### Colon Cancer

In one aspect, compounds of the invention may be used for the treatment of colon cancer, including but not limited to non-neoplastic polyps, adenomas, familial syndromes, colorectal carcinogenesis, colorectal carcinoma, and carcinoid tumors. Possible treatments available for colon cancer that may be used in conjunction with the compounds of the invention include surgery, chemotherapy, radiation therapy or targeted drug therapy.

Radiation therapy may include external beam radiation therapy and/or brachytherapy. Chemotherapy may be used to reduce the likelihood of metastasis developing, shrink tumor size, or slow tumor growth. Chemotherapy is often applied after surgery (adjuvant), before surgery (neo-adjuvant), or as the primary therapy if surgery is not indicated (palliative). For example, exemplary regimens for adjuvant chemotherapy involve the combination of infusional 5-fluorouracil, leucovorin, and oxaliplatin (FOLFOX). First line chemotherapy regimens may involve the combination of infusional 5-fluorouracil, leucovorin, and oxaliplatin (FOLFOX) with a targeted drug such as bevacizumab, cetuximab or panitumumab or infusional 5-fluorouracil, leucovorin, and irinotecan (FOLFIRI) with targeted drug such as bevacizumab, cetuximab or panitumumab. Other chemotherapeutic agents that may be useful in the treatment or prevention of colon cancer in combination with the compounds of the invention are Bortezomib (Velcade®), Oblimersen (Genasense®, G3139), Gefitinib and Erlotinib (Tarceva®) and Topotecan (Hycamtin®).

### Lung Cancer

Some embodiments provide methods for the treatment of lung cancer using the compounds of the invention. Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer, e.g. small cell lung carcinomas, accounts for 15-20% of lung cancers. Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy. Some anti-cancer drugs that may be used in chemotherapy to treat lung cancer in combination with the compounds of the invention include cisplatin, carboplatin, paclitaxel, docetaxel, gemcitabine, vinorelbine, irinotecan, etoposide, vinblastine, gefitinib, ifosfamide, methotrexate, or a combination thereof. Photodynamic therapy (PDT) may be used to treat lung cancer patients. The methods described herein can provide a beneficial effect for lung cancer patients, by administration of a compound or a combination of administration of a compound and surgery, radiation therapy, chemotherapy, photodynamic therapy, or a combination thereof.

Examples of cellular proliferative and/or differentiative disorders of the liver include, but are not limited to, nodular hyperplasias, adenomas, and malignant tumors, including primary carcinoma of the liver and metastatic tumors.

### Immunoproliferative Disorders

Immunoproliferative disorders (also known as “immunoproliferative diseases” or “immunoproliferative neoplasms”) are disorders of the immune system that are characterized by the abnormal proliferation of the primary cells of the immune system, which includes B cells, T cells and Natural Killer (NK) cells, or by the excessive production of immunoglobulins (also known as antibodies). Such disorders include the general categories of lymphoproliferative disorders, hypergammaglobulinemias, and paraproteinemias. Examples of such disorders include, but are not limited to, X-linked lymphoproliferative disorder, autosomal lymphoproliferative disorder, Hyper-IgM syndrome, heavy chain disease, and cryoglobulinemia. Other immunoproliferative disorders can be graft versus host disease (GVHD); psoriasis; immune disorders associated with graft transplantation rejection; T cell lymphoma; T cell acute lymphoblastic leukemia; testicular angiocentric T cell lymphoma; benign lymphocytic angiitis; and autoimmune diseases such as lupus erythematosus, Hashimoto's thyroiditis, primary myxedema, Graves' disease, pernicious anemia, autoimmune atrophic gastritis, Addison's disease, insulin dependent diabetes mellitis, good pasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulceratis colitis, Sjogren's syndrome, rheumatoid arthritis, polymyositis, scleroderma, and mixed connective tissue disease.

### Combination Treatments

In one embodiment, compounds of the invention may be used for the treatment of cancer in conjunction with alkylating and alkylating-like agents. Such agents include, for example, nitrogen mustards such as chlorambucil, chlormethine, cyclophosphamide, ifosfamide, and melphalan; nitrosoureas such as carmustine, fotemustine, lomustine, and streptozocin; platinum therapeutic agents such as carboplatin, cisplatin, oxaliplatin, BBR3464, and satraplatin; or other agents, including but not limited to busulfan, dacarbazine, procarbazine, temozolomide, thiotepa, treosulfan, or uramustine.

In another embodiment, compounds of the invention may be used in conjunction with an antineoplastic agent which is an antimetabolite. For example, such an antineoplastic agent may be a folic acid such as aminopterin, methotrexate, pemetrexed, or raltitrexed. Alternatively, the antineoplastic agent may be a purine, including but not limited to cladribine, clofarabine, fludarabine, mercaptopurine, pentostatin, thioguanine In further embodiments, the antineoplastic agent may be a pyrimidine such as capecitabine, cytarabine, fluorouracil, floxuridine, and gemcitabine.

In still other embodiments, compounds of the invention may be used in conjunction with an antineoplastic agent which is an spindle poison/mitotic inhibitor. Agents in this category include taxanes, for example docetaxel and paclitaxel; and vinca alkaloids such as vinblastine, vincristine, vindesine, and vinorelbine. In yet other embodiments, compounds of the invention may be used in combination with an antineoplastic agent which is a cytotoxic/antitumor antibiotic from the anthracycline family such as daunorubicin, doxorubicin, epirubicin, idarubicin, mitoxantrone, pixantrone, or valrubicin; an antibiotic from the streptomyces family such as actinomycin, bleomycin, mitomycin, or plicamycin; or hydroxyurea. Alternatively, agents used for combination therapy may be topoisomerase inhibitors including, but not limited to camptothecin, topotecan, irinotecan, etoposide, or teniposide.

Alternatively, the antineoplastic agent may be an antibody or antibody-derived agent. For example, a receptor tyrosine kinase-targeted antibody such as cetuximab, panitumumab, or trastuzumab may be used Alternatively, the antibody may be an anti-CD20 antibody such as rituximab or tositumomab, or any other suitable antibody including but not limited to alemtuzumab, bevacizumab, and gemtuzumab. In other embodiments, the antineoplastic agent is a photosensitizer such as aminolevulinic acid, methyl aminolevulinate, porfimer sodium, or verteporfin. In still other embodiments, the antineoplastic agent is a tyrosine kinase inhibitor such as dediranib, dasatinib, erlotinib, gefitinib, imatinib, lapatinib, nilotinib, sorafenib, sunitinib, or vandetanib. Other neoplastic agents suitable in the use of the invention include, for example, alitretinoin, tretinoin, altretamine, amsacrine, anagrelide, arsenic trioxide, asparaginase (pegaspargase), bexarotene, bortezomib, denileukin diftitox, estramustine, ixabepilone, masoprocol, or mitotane.

In other or further embodiments, the compounds described herein are used to treat, prevent or diagnose conditions characterized by overactive cell death or cellular death due to physiologic insult, etc. Some examples of conditions characterized by premature or unwanted cell death are or alternatively unwanted or excessive cellular proliferation include, but are not limited to hypocellular/hypoplastic, acellular/aplastic, or hypercellular/hyperplastic conditions. Some examples include hematologic disorders including but not limited to fanconi anemia, aplastic anemia, thalaessemia, congenital neutropenia, and myelodysplasia.

In other or further embodiments, the compounds of the invention that act to decrease apoptosis are used to treat disorders associated with an undesirable level of cell death. Thus, in some embodiments, the anti-apoptotic compounds of the invention are used to treat disorders such as those that lead to cell death associated with viral infection, e.g., infection associated with infection with human immunodeficiency virus (HIV). A wide variety of neurological diseases are characterized by the gradual loss of specific sets of neurons, and the anti-apoptotic compounds of the invention are used, in some embodiments, in the treatment of these disorders. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

### Other Methods of Use

In other or further embodiments, the anti-apoptotic compounds of the invention are used to treat all such disorders associated with undesirable cell death.

Some examples of immunologic disorders that are treated with the compounds described herein include but are not limited to organ transplant rejection, arthritis, lupus, IBD, Crohn's disease, asthma, multiple sclerosis, diabetes, etc.

Some examples of neurologic disorders that are treated with the compounds described herein include but are not limited to Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a prion-mediated disease, and Huntington's Disease.

Some examples of endocrinologic disorders that are treated with the compounds described herein include but are not limited to diabetes, hypothyroidism, hypopituitarism, hypoparathyroidism, hypogonadism, etc.

Examples of cardiovascular disorders (e.g., inflammatory disorders) that are treated or prevented with the compounds of the invention include, but are not limited to, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices. Preferred cardiovascular disorders include atherosclerosis, myocardial infarction, aneurism, and stroke.

### EXAMPLES

### Example 1

### Compound Preparation

General. Commercial-grade reagents and solvents were used without further purification, except as indicated. Dry DMF was obtained using an Innovative Technology PureSolv solvent drying system. All reactions were either stirred or mechanically shaken at room temperature, except as indicated. After each step, the resin was sequentially washed with DMF (3×5 mL), MeOH (3×5 mL), and DCM (3×5 mL). Microwave irradiation was performed in the CEM Discover single-mode reactor with controlled power, temperature, time and stirring settings. NMR experiments were performed using a Bruker AVANCE 500 MHz spectrometer. Reversed-phase HPLC experiments were conducted with 4.6×150 mm (analytical scale) or 21.4×150 mm (preparative scale) Waters C18 Sunfire columns using a Beckman Coulter HPLC equipped with a System Gold 168 Diode array detector. HPLC buffers consisted of 0.1% aqueous trifluoroacetic acid and 0.1% trifluoroacetic acid in acetonitrile. Mass spectra were obtained by either an Agilent 1100 series LC/MSD (XCT) electrospray trap or a Bruker UltraflexXtreme MALDI-TOF/TOF. The peptide concentrations for the CD and NMR studies were calculated using absorbance of the tryptophan residue (5560 cm⁻¹ M⁻¹ at 280 nm).

Synthesis of bis-thiol precursor peptide 1. Parent peptide 3 (0.25 mmol) was synthesized on a CEM Liberty microwave peptide synthesizer using standard Fmoc solid phase chemistry with Knorr Amide MBHA resin. The resin bearing 3 was transferred to a fritted polypropylene SPE tube, washed, and dried under vacuum. A solution of preactivated bromoacetic acid (0.17 g, 1.25 mmol, 5 equiv), HOBt (0.18 g, 1.25 mmol, 5 equiv), DIC (0.20 mL, 1.25 mmol, 5 equiv) in 3 mL dry DMF was added to the resin containing 3. After 3 h, the resin was washed and treated with S-trityl-2-mercaptoethylamine (0.45 g, 1.25 mmol, 5 equiv) and DIEA (0.66 mL, 3.37 mmol, 15 equiv) in 3 mL dry DMF. After 3 h, the resin containing 4 was washed. The resin was dried under vacuum for 1 h, transferred to a microwave tube, capped, and purged with N₂ for 1 h.

To the microwave tube containing dried 4 on resin, a solution of preactivated Fmoc-Glu(tBu)-OH (2.13 g, 5.0 mmol, 20 equiv), HOAt (0.35 g, 2.5 mmol, 10 equiv), DIC (0.66 mL, 5.0 mmol, 20 equiv) in 4 mL dry DMF was added. The reaction mixture was subjected to microwave irradiation at 60° C. for 45 min, after which the resin was transferred to an SPE tube and washed. The resin was treated with 20% piperidine in NMP (2×20 min) for Fmoc deprotection, washed and dried under vacuum. A solution of preactivated Fmoc-Gln(Trt)-OH (0.76 g, 1.25 mmol, 5 equiv), HBTU (0.42 g, 1.13 mmol, 4.5 equiv) in 4.1 mL 5% DIEA/NMP was added to the resin. After 3 h, the resin containing 5 was washed and dried under vacuum.

Resin bound 5 was subjected to Fmoc deprotection and bromoacetylation as described above. A solution of triphenylmethyl mercaptan (0.35 g, 1.25 mmol, 5 equiv) and DIEA (0.66 mL, 3.37 mmol, 15 equiv) in 3 mL dry DMF was added to the resin. After 3 h, the resin was washed and dried under vacuum. The dried resin was treated with 81.5% TFA, 5% H₂O, 5% thioanisole, 5% phenol, 2.5% EDT, 1% TIPS. After 2 h, the mixture was filtered and concentrated in vacuo. The crude solid was washed with cold ether and dried under N₂. HPLC purification (gradient of 5-75 acetonitrile/water in 45 min) and lyophilization yielded bis-thiol peptide 1 as a white powder (10 mg). [M+H]⁺ calculated: 1555.73; [M+H]⁺ observed: 1555.71; Analytical HPLC Rₜ: 9.3 min (gradient of 5-95 acetonitrile/water in 15 min).

Synthesis of dsHBS 2. Bis-thiol peptide 1 (5 mg) was dissolved in 6.3 mL of 0.1 M aqueous ammonium bicarbonate solution (buffered to pH 6 with TFA) containing 20% DMSO and 10% TFE. After 15 h, the mixture was frozen and lyophilized to obtain a colorless oil. HPLC purification (gradient of 5-65 acetonitrile/water in 45 min) and lyophilization yielded dsHBS 2 (1 mg, 20%) as a white powder. [M+H]⁺ calculated: 1553.71; [M+H]⁺ observed: 1553.72; Analytical HPLC Rₜ: 9.5 min (gradient of 5-95 acetonitrile/water in 15 min).

Synthesis of S-trityl-mercaptoethylamine hydrochloride. Cysteamine hydrochloride (1.00 g, 8.8 mmol, 1 equiv) was dissolved in DMF-DCM (1:1, 50 mL), followed by the addition of trityl chloride (3.68 g, 13.2 mmol, 1.5 equiv). After 3 h, insoluble material was filtered and washed with DCM (3×10 mL). The crude product was concentrated in vacuo and subsequently re-constituted and re-concentrated (3×50 mL DCM). Purification by flash chromatography (10% MeOH/DCM) yielded 2.35 g of off-white solid (75% yield). R_{f}: 0.60 (1:9 MeOH/DCM). ¹H NMR (CDCl₃): □=2.36-2.60 (m, 4H), 5.25 (broad s, 3H), 7.19-7.56 (m, 15H). ¹³C NMR (CDCl₃): □=32.08, 39.57, 67.05, 126.88, 128.08, 129.53, 144.44.

### Example 2

### Circular Dichroism Spectroscopy

CD spectra were recorded on an AVIV 202SF CD spectrometer equipped with a temperature controller using 1 mm length cells and a scan speed of 5 nm/min. The spectra were averaged over 10 scans with the baseline subtracted from analogous conditions to those of the samples. The samples were prepared in 0.1× phosphate buffered saline (13.7 mM NaCl, 1 mM phosphate, 0.27 mM KCl, pH 7.4), with the final peptide concentration of 50 μM. To monitor the effects of a reducing agent on the helix conformation, a sample of dsHBS 2 was prepared with 75 □M TCEP in the buffer conditions; reduction of the disulfide bond to bis-thiol was confirmed by mass spectroscopy. The helix content of each peptide was determined from the mean residue CD at 222 nm, [θ]₂₂₂ (deg cm² dmol⁻¹) corrected for the number of amino acids. Percent helicity was calculated from the ratio [θ]₂₂₂/[θ]ₘₐₓ, where [θ]ₘₐₓ=(−44000+250T)(1−k/n)=−25,170 for T=25 (° C.), k=4.0, and n=12 (number of amino acid residues).^{7,20,23}

### Example 3

### 2D NMR Spectroscopy

Spectra of dsHBS 2 were recorded on a Bruker Avance 500 at 20° C. The sample was prepared by dissolving 1 mg of 2 in 300 □L of 20% TFE-d₃ in PBS (pH 3.5) in a Shigemi NMR tube. All 2D spectra were recorded by collecting 2048 complex data points in the t2 domain by averaging 72 scans and 512 increments in the t1 domain with the States-TPPI mode. TOCSY experiments were performed with a mixing time of 80 ms on a 6000 Hz spin lock frequency. For NOESY, mixing time of 200 ms was used. The data were processed and analyzed using the Bruker TOPSPIN program. The original free induction decays (FIDs) were zero-filled to give a final matrix of 1024 by 1024 real data points. A 90° sine-square window function was applied in both dimensions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of synthesizing a stabilized helical peptidomimetic macrocycle, said method comprising:
providing a peptidomimetic precursor comprising two thiol groups; and
contacting the precursor with a reagent capable of inducing a reaction between said two thiol groups, said reaction resulting in formation of a disulfide covalent bond;
wherein said contacting step results in cyclization of the precursor to form said stabilized helical peptidomimetic macrocycle, and
wherein said stabilized helical peptidomimetic macrocycle comprises a structure of formula: 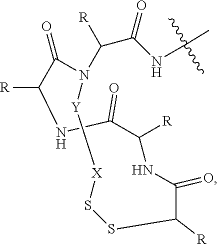
wherein each R is independently an amino acid side chain and X—Y is a crosslinker moiety.

2. The method of claim 1, wherein the peptidomimetic macrocycle has higher α-helicity compared to a corresponding non-macrocyclic polypeptide.

3. The method of claim 1, wherein the peptidomimetic macrocycle has higher α-helicity compared to the peptidomimetic precursor.

4. The method of claim 2, wherein the α-helicity is measured by circular dichroism.

5. The method of claim 1, wherein the peptidomimetic macrocycle exhibits increased resistance to proteolytic degradation compared to a corresponding non-macrocyclic polypeptide.

6. The method of claim 1, wherein the peptidomimetic macrocycle exhibits increased biological activity compared to a corresponding non-macrocyclic polypeptide.

7. The method of claim 1, wherein the peptidomimetic precursor is prepared by solid phase peptide synthesis resin.

8. The method of claim 1, wherein the peptidomimetic precursor is attached to a solid phase peptide synthesis resin during the contacting step.

9. The method of claim 1, wherein the peptidomimetic precursor is not attached to a solid phase peptide synthesis resin during the contacting step.

10. The method of claim 1, wherein the contacting step takes place in a solvent.

11. The method of claim 10, wherein the solvent is an aqueous solvent.

12. The method of claim 11, wherein the solvent comprises DMSO.

13. The method of claim 11, wherein the solvent comprises TFE.

14. The method of claim 1, wherein the peptidomimetic macrocycle is purified after the contacting step.

15. The method of claim 1, wherein the stabilized helical peptidomimetic macrocycle comprises a structure of formula: 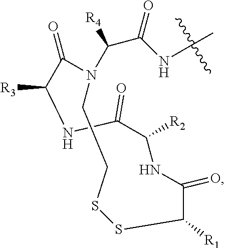
wherein R₁, R₂, R₃, and R₄ are each independently an amino acid side chain.

16. The method of claim 1, wherein the stabilized helical peptidomimetic macrocycle comprises a structure of formula: 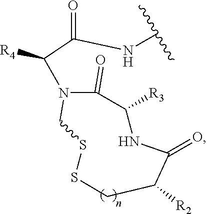
wherein R₁, R₂, R₃ and R₄ are each independently an amino acid side chain.

17. A peptidomimetic macrocycle comprising a structure of formula: 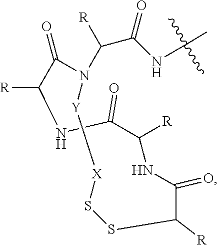
wherein each R is independently an amino acid side chain and X—Y is a crosslinker moiety.

18. The peptidomimetic macrocycle of claim 17, wherein the peptidomimetic macrocycle comprises a structure of formula: 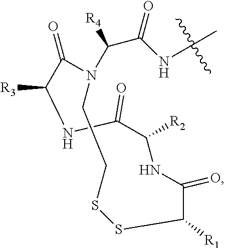
wherein R₁, R₂, R₃, and R₄ are each independently an amino acid side chain.

19. The peptidomimetic macrocycle of claim 17, wherein the peptidomimetic macrocycle comprises a structure of formula: 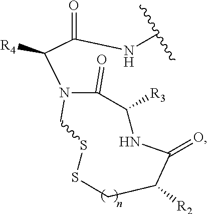
wherein R₁, R₂, R₃, and R₄ are each independently an amino acid side chain.

20. The method of claim 3, wherein the α-helicity is measured by circular dichroism.
